(19) United States
(12) Patent Application Publication
(10) Pub. No.: US 2025/0381045 A1
(43) Pub. Date: Dec. 18, 2025

(54) METHOD, COMPUTER PROGRAM, AND SYSTEM FOR ADJUSTING AN ORTHOPEDIC DEVICE

(71) Applicant: OTTO BOCK HEALTHCARE PRODUCTS GMBH, Wien (AT)

(72) Inventors: Serhii ANDRUSHCHENKO, Wien (AT); Thomas HOFMANN, Wien (AT); Paul PEYER, Wien (AT)

(21) Appl. No.: 18/878,393

(22) PCT Filed: Jun. 30, 2023
(86) PCT No.: PCT/EP2023/068026

§ 371 Date: Dec. 23, 2024

(30) Foreign Application Priority Data
Jul. 1, 2022 (DE) 10 2022 116 496.9

(51) Int. Cl. A61F 2/70; A61F 2/64

(52) U.S. Cl. CPC A61F 2/70; A61F 2/64; A61F 2002/704; A61F 2002/705

(57) ABSTRACT

The invention relates to a method for adjusting a microprocessor-controlled orthopedic device which is worn on the body of a patient fitted with such a device and influences movement by microprocessor-controlled actuation of actuators, the method comprising the following computer-implemented steps:
providing a digital communication link from a mobile terminal device to an adjustment database, in which at least one adjustment value for at least one parameter of the respective microprocessor-controlled actuation and associated patient data of a patient fitted with the respective orthopedic device are stored for a plurality of microprocessor-controlled orthopedic devices,
calculating at least one adjustment value of at least one parameter of the microprocessor-controlled actuation of the orthopedic device of the patient depending on patient data from the patient whose microprocessor-controlled orthopedic device is to be adjusted, and/or the adjustment values stored in the adjustment database and associated patient data,
displaying the at least one calculated adjustment value on the mobile terminal device in order to adjust the microprocessor-controlled orthopedic device.

The invention relates to a method, a computer program and a system for adjusting a microprocessor-controlled orthopedic device which is worn on the body of a patient fitted with such a device and influences movement by microprocessor-controlled actuation of actuators.

Within the context of the present invention, orthopedic devices refer, in particular, to orthoses, prostheses, exoskeletons and possibly wheelchairs. Orthoses are products that brace, support, protect or restrict the freedom of movement of a body part of the patient in order to prevent excessive strain. Conversely, prostheses replace body parts that are missing or are no longer there. Specifically, exoskeletons are mechanical support structures that serve to brace, support or protect the patient's main musculoskeletal system.

In the following, the term patient refers to any user of the orthopedic device. It is therefore the person wearing the orthopedic device.

Orthopedic devices, such as orthoses, prostheses or collars, wheelchairs, exoskeletons and the like, are used individually. Orthopedic devices may have power units, locks, brakes and/or dampers that are activated or deactivated via an appropriately set control device. The control device may be connected to one or multiple sensors in order to modify the behavior of the orthopedic device on the basis of sensor data.

On the basis of the sensor data and the adjustment values with which the orthopedic device was configured (adjusted), the actuators of the orthopedic device are actuated in a microprocessor-controlled manner to induce the desired behavior of the orthopedic device.

U.S. Pat. No. 6,679,920 B2 discloses a device and a method for remote maintenance of an electronically controlled prosthesis. The prosthesis is in the form of a prosthetic leg fitted with a prosthesis socket, a prosthetic knee, a lower leg section and a prosthetic foot. A hydraulic damper is arranged between the prosthesis socket and the lower leg section, the former adjusting the damping of the hydraulic damper on the basis of forces or accelerations, for example. A separate maintenance device or docking station can be connected to the control device of the prosthesis. The maintenance device collects the control and movement data of the prosthesis, analyses it, processes it and, where relevant, provides the prosthesis control device with an updated data set. Data transmission can be done wirelessly via a data network or a telephone line.

U.S. Pat. No. 10,015,840 B2 relates to a device and a method in which sensor data of a unit that can be worn separately is collected and compared with a movement model. If the model does not match the sensor data, correction orders are given. As soon as sufficient correction has occurred, the correction process is terminated.

US 2005/107726 relates to a method for monitoring an orthosis from a distance. In particular, a healthcare professional is audio-visually connected to a user of an orthosis who uses the orthosis to perform exercises following injury or similar. At the same time, data from the orthosis, for example from angle or force sensors, is transmitted to the healthcare professional. The aim of this is to monitor the correct performance of the exercises. Patient data that is decoupled from personal data is entered into a database in order, for example, to compare and, where necessary, to improve the effectiveness of a therapeutic approach in multiple similar cases.

US 2008/103506 A1 relates to a wearable unit in the form of an orthosis that can even be used for someone recovering from a stroke or similar. Sensors are used to identify conditions, which are then sent to a computer or a network-based device. The purpose of this is to recognize a movement that deviates from an intended movement and to provide the user with corrective instructions.

EP 2 153 370 B1 discloses a system for aligning prostheses in which both movement data of the person fitted with the prosthesis and prosthesis alignment errors are determined from a movement database. Said data is compared to ascertain whether the prosthesis corresponds to a target value or has to be adjusted further.

DE 10 2012 009 507 A1 contains a method and a device for determining misalignments in the structure of lower limb prostheses, wherein inertial measurement data is determined over at least one gait cycle and compared with target values.

DE 10 2018 128 514 B4 discloses a method for carrying out a static prosthesis assembly of a prosthesis, wherein several components are arranged next to each other. In the process, an actual position and an actual orientation of the components arranged next to each other relative to each other are determined using the detected positions and orientations of markers and compared with the corresponding target values.

The adjustment of microprocessor-controlled orthopedic devices with the corresponding adjustment values, which ensure optimal adjustment and treatment of the patient with the necessary orthopedic device, usually depends on the experience of the orthopedic technician in order to achieve good quality treatment. The best possible adjustment is sought in an iterative process, in which the patient uses the orthopedic device in everyday life after each adjustment by the orthopedic technician and reports this back to the orthopedic technician. This adjustment process is very time-consuming and cost-intensive, especially for orthopedic technicians with little experience.

The present invention therefore aims to present an improved method for adjusting an orthopedic device.

The task is solved according to the invention using the method specified above by way of the features of patent claim 1. Advantageous embodiments of the invention are then to be found in the corresponding sub-claims.

According to claim 1, a method for adjusting a microprocessor-controlled orthopedic device which is worn on the body of a patient fitted with such a device and influences movement by microprocessor-controlled actuation of actuators is proposed, the method comprising the following computer-implemented steps:
providing a digital communication link from a mobile terminal device to an adjustment database, in which at least one adjustment value for at least one parameter of the respective microprocessor-controlled actuation and associated patient data of a patient fitted with the respective orthopedic device are stored for a plurality of microprocessor-controlled orthopedic devices,
calculating at least one adjustment value of at least one parameter of the microprocessor-controlled actuation of the orthopedic device of the patient depending on patient data from the patient whose microprocessor-controlled orthopedic device is to be adjusted, and/or the adjustment values stored in the adjustment database and associated patient data,
displaying the at least one calculated adjustment value on the mobile terminal device in order to adjust the microprocessor-controlled orthopedic device.

The method according to the invention initially comprises providing a digital communication link from a mobile terminal device to an adjustment database. The mobile terminal device can be, for example, a smartphone, tablet, laptop or a PC. The digital communication link is preferably provided with the aid of a standardized interface, for example via a wired and/or wireless communication link (TCP/IP, GSM, UMTS, LTE, etc.), and, in particular, is a network-based communication link.

For a large number of microprocessor-controlled orthopedic devices, the adjustment database contains at least one adjustment value for at least one parameter for the microprocessor-controlled actuation of the orthopedic device in conjunction with the associated patient data of the patient who is fitted with the specific orthopedic device. This means that, for an orthopedic device, the patient data and the adjustment values of the orthopedic device for its actuation are stored in the database.

Via a communication interface in the mobile terminal device, the mobile terminal device can access this adjustment database with the aid of the digital communication link.

If a specific orthopedic device is to be adjusted for a specific patient, the optimum adjustment values for at least one parameter of the microprocessor-controlled actuation of the orthopedic device to be adjusted are determined from the adjustment database, depending on the patient data of this patient and the orthopedic device used.

To do this, the adjustment values of other orthopedic devices matching the orthopedic device to be adjusted are first determined from the adjustment database as well as the corresponding patient data. On the basis of the patient data of the patient whose orthopedic device is to be adjusted, the optimal adjustment values of the parameters of the orthopedic device to be adjusted are calculated from the determined adjustment values from the adjustment databased and the associated patient data. This can be done algorithmically, for example, or using a previously learnt knowledge database (an AI, for example).

The calculation of the adjustment values for the orthopedic device can be performed using the adjustment database, for example. The adjustment database comprises a data processing system, in which, in addition to the actual database with the adjustment values and patient data, the calculation of the adjustment values of the orthopedic device to be adjusted is also carried out. Data capacities can be saved here, particularly in the case of wireless transmission, wherein the execution speed on a central data processing system may be more optimal than on a low-performance terminal device.

However, it is also conceivable that the adjustment values and the associated patient data are transferred from the adjustment database to the mobile terminal device, wherein only the adjustment values and patient data that are also included in the calculation are transmitted. It is therefore not necessary to transfer the entire content of the database. On the basis of the adjustment values and patient data transmitted, the calculations are now performed on the mobile terminal device so as to calculate the adjustment values of at least one parameter for the orthopedic device to be adjusted.

Preferably, at least one adjustment value is calculated for a plurality of parameters of the microprocessor-controlled actuator of the orthopedic device to be adjusted. It can also be provided for that multiple adjustment values are calculated for one parameter of the microprocessor-controlled actuation, for example in order to provide an optimal range within which the adjustment values are all considered to be optimal or to provide alternative adjustments of the orthopedic device in connection with other adjustment values.

In particular, an adjustment of the orthopedic device includes one or multiple parameters, each with at least one adjustment value.

Once the adjustment values for the patient's orthopedic device to be adjusted have been calculated, the adjustment values are displayed on the mobile terminal device. These displayed adjustment values, which have been calculated beforehand, are now used to adjust the microprocessor-controlled orthopedic device of the corresponding patient.

The present invention provides the advantage that the adjustment process becomes time-saving and more efficient, even if the orthopedic technician has little experience. This shortens interaction steps, thereby also increasing the patient's comfort. Furthermore, it allows access to the full potential of the experience of other orthopedic technicians, as the adjustment values stored in the database can be traced back to such experience.

A further advantage of the present invention is that the adjustment values become increasingly precise in the manner of a self-learning system when the adjustment values individually adjusted on the respective patient are transmitted back to the adjustment database. Over time, this results in an ever-growing number of individual adjustment values in connection with the respective patient data, which is connected to the respective adjustment values, so that the adjustment values for a particular patient can be calculated increasingly precisely. The calculation is therefore a function of time and the number of incoming parameters.

It is conceivable that not only orthopedic devices on one side of the body are taken into account, but also orthopedic devices on both sides of the body. The adjustment values of the orthopedic device on the first half of the body can influence the adjustment values of the orthopedic device on the second half of the body so as to obtain an optimal result. This means that when determining the adjustment values of the first half of the body, adjustment values of the second half of the body can also be taken into account for patients with a bilateral device.

According to one embodiment, it is provided for, that the patient data of the patient whose microprocessor-controlled orthopedic device is to be adjusted is transmitted from the mobile terminal device to the adjustment database, wherein the at least one adjustment value of the at least one parameter is calculated by the adjustment database.

According to one embodiment, it is intended that the at least one adjustment value is calculated as a function of the stored adjustment values that relate to the same microprocessor-controlled orthopedic device or at least the same type or kind of device.

In this way, the data stored in the adjustment database can be filtered to those data that relate to the orthopedic device to be adjusted and whose parameters, for which the adjustment values are stored in the database, correspond to those of the orthopedic device to be adjusted.

According to one embodiment, it is intended that the at least one adjustment value is calculated as a function of the stored adjustment values that are no older than a given date.

With this embodiment, only adjustment values are taken into account which, starting from the current date, are no older than a certain time period, so as to only take adjustment values into account that exhibit a certain up-to-datedness or whose initial configuration is no older than the given date.

According to one embodiment, it is provided for that at least one correlation parameter is calculated by means of the adjustment database and transmitted to the mobile terminal device, wherein at least one adjustment value is calculated as a function of the patient data and the at least one correlation parameter, even without an existing communication link.

The at least one correlation parameter—preferably multiple correlation parameters are used—refers to a weighting of individual parameters that are included in the calculation of the individual adjustment values. The correlation parameter or parameters are saved on the mobile terminal device, only being updated when a new communication link has been established.

According to one embodiment of this, it is intended that the at least one adjustment value is calculated as a function of the stored adjustment values where a change was made by an orthopedic professional that is no older than a given date.

In this embodiment, only adjustment values are taken into account which have been amended by an orthopedic professional in the recent past, so that in this case too, only current or modified adjustment values are taken into account.

By selecting the appropriate date, and the adjustment values must not be older, it is also possible to take into account how long the patient has already had the orthopedic device to be adjusted. This is because, in general, wearing an orthopedic device can cause the parameters of the patient's body to change in the area of the orthopedic device, meaning that a readjustment is necessary. This can be taken into account by filtering the data from the adjustment database accordingly.

Therefore, a core idea of the present invention is also that only those adjustment values from the adjustment database are taken into account for the calculation which are older than a given date in order to filter for optimally adjusted data, even when the orthopedic device has already been in use for a longer period of time.

According to one embodiment, it is intended that the at least one adjustment value is calculated as a function of the stored adjustment values that relate to a microprocessor-controlled orthopedic device that has performed a particular number of movements.

This ensures that only adjustment values are taken into account that, through a large number of movements, have proven advantageous for the patient. This is because adjustment values that only record a small number of movements of the associated orthopedic device are often perceived as disturbing for the patient and lead to an adjustment and thus to the values being overwritten.

According to one embodiment, it is intended that, after adjusting the patient's microprocessor-controlled orthopedic device, the current adjustment values are transmitted from the mobile terminal device to the adjustment database.

As a result, the newly calculated adjustment values and/or, where applicable, those that have been slightly adjusted by the orthopedic technician can also be entered into the database along with the patient data of the patient whose orthopedic device is to be adjusted, so that they can be taken into account during the calculation for other patients. This allows the database to be kept up to date over time and approaches an optimum for the respective patient with the corresponding patient data.

According to one embodiment, it is intended that the adjustment of the patient's microprocessor-controlled orthopedic device with the up-to-date adjustment values is performed by means of the mobile terminal device, which establishes a communication link to the microprocessor-controlled orthopedic device to do so.

The calculated adjustment values, which are displayed on the mobile terminal device and, where applicable, have been slightly readjusted by the orthopedic technician, are then transmitted The entire adjustment process can thus be carried out starting from the communication link provided to the adjustment database, through the calculation of the adjustment values, to the communication link with the orthopedic device and the adjustment of the device using the mobile terminal device, in particular without a disruption in media in the entire adjustment process.

According to one embodiment, it is intended that a machine learning system (as part of the adjustment database) is provided which has learned a correlation of patient data of patients and information about their respective microprocessor-controlled orthopedic devices as input data with associated adjustment values of at least one parameter as output data, wherein the patient data of the patient and information on their microprocessor-controlled orthopedic device is entered into the machine learning system as input data and at least one calculated adjustment value is subsequently obtained as output data in order to calculate the at least one adjustment value for adjusting the patient's device.

Preferably, such a machine learning system is an artificial neural network.

The machine learning system has learned a correlation between patient data of the patient and information about their orthopedic device with adjustment values for this orthopedic device. The machine learning system forms part of the adjustment database or part of the calculation.

In this case, the training data originates from the adjustment values stored in the adjustment database along with the associated patient data and information about the orthopedic device to which the adjustment values and the patient data belongs. A training data set thus contains information on the orthopedic device and a selection of patient data (such as age, weight, duration of treatment etc.) as well as the specific adjustment values of this orthopedic device. Such a training data set is generated for each patient with an orthopedic device such that, through the sum of patients, a variety of such training data sets is available with which the machine learning system can then be trained in the known manner.

The idea of the invention likewise includes a computer program with program coding means configured to carry out the method described above when the computer program is run on a data processing system.

The idea of the invention also includes a system comprising an adjustment database and one or multiple mobile terminal devices, wherein the system is configured to carry out the method described above. A data processing system may be provided on which the adjustment database is executed. This data processing system can also be used to perform the calculation for specific adjustment values and, if necessary, the machine-learning system, which performs an AI-based calculation.

The invention is explained in more detail by means of the attached figure. It shows:

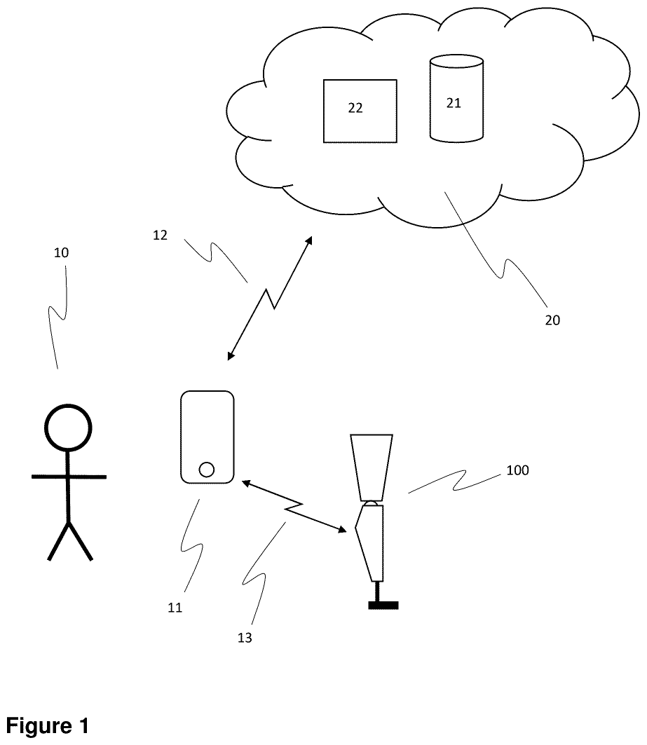

FIG. 1 schematic representation of the system and method according to the invention

In a schematically simplified illustration, FIG. 1 shows an orthopedic technician 10 or an orthopedic professional who is operating a mobile terminal device 11 for adjusting a microprocessor-controlled orthopedic device 100. This orthopedic device 100 is an orthopedic device of a patient that is not depicted.

Patient data of the patient who is to use the orthopedic device 100 is stored on the terminal device 11. Such patient data can be stored locally on the terminal device 11 or, where possible, network-compatible databases (cloud). Furthermore, information on the orthopedic device 100 is stored on the terminal device 11 in order to make it distinguishable in terms of use, type and production series.

The patient data may contain, for example, the height and weight of the patient as well as their impairment that is to be addressed by the orthopedic device 100. In addition, the patient data may also include the patient's age and possibly a movement profile, i.e. whether the patient is very active or tends to move less. Medical conditions and the patient's history of impairment or other diseases may also form part of the patient data that is taken into account when determining the adjustment values for the orthopedic device 100.

Both the patient data and the information on the orthopedic device 100 are transmitted by the orthopedic technician 10 by means of the terminal device 11 to a data processing system 20 that can be reached via a communication link 12 of a network.

The data processing system comprises an adjustment database 21 and a calculation unit 22. With the aid of the patient data and the information on the orthopedic device 100 whose parameters are to be adjusted, those adjustment values from the adjustment database 21 that correspond with the orthopedic device 100 are determined. This means that only the data from the adjustment database is taken into account for which the adjustment values and patient data belong to an orthopedic device that corresponds to the orthopedic device 100 to be adjusted.

In the embodiment in FIG. 1, an orthopedic device 100 in the form of a knee prosthesis is to be adjusted in which an artificial knee is provided that can be adjusted by way of relevant adjustment parameters with regard to its behavior. In addition, only those adjustment values are determined from database 21 that also relate to a knee prosthesis with an artificial knee of the intended type.

Further filter parameters can be provided, such as the date of the initial configuration and/or the date of the most recent modification. This ensures that up-to-date data is always available.

From these adjustment values determined from the adjustment database 21 for various orthopedic devices in the field, an adjustment value is now determined for at least one parameter of the orthopedic device 100, taking into account the patient data of the patient to be treated and the patient data in the database belonging to the adjustment values. Preferably, however, multiple parameters are determined for the orthopedic device 100.

The calculation of the adjustment values for the orthopedic device 100 is performed with the aid of the calculation unit 22. In one embodiment, the calculation unit 22 can form a common unit integrally with the adjustment database 21, in which a machine learning system (for example an AI) is provided, in which both the calculation and the data storage form an integral unit.

By entering the information on the orthopedic device 100 as well as the patient data, the adjustment values can be determined for one or multiple parameters of the orthopedic device 100 on the basis of the learned correlation. It can be provided for that different types of orthopedic device are each provided with their own machine learning system.

The adjustment values determined in this way, which have been determined by the data processing system 20, are then transmitted to the terminal device 11, where they are shown on a display. These adjustment values shown on the display are now used to adjust the orthopedic device 100 accordingly.

For this purpose, it may be provided that a further communication link 13 is established from the mobile terminal device 11 to the orthopedic device 100 in order to electronically transfer the determined adjustment values, which may have been adjusted by the orthopadic technician 10, to the orthopedic device 100.

### REFERENCE LIST

10 orthopedic technician
11 mobile end terminal
12 communication link to the database
13 communication link to the orthopedic device
20 data processing system
21 adjustment database
22 calculation unit
100 orthopedic device

1. A method for adjusting a microprocessor-controlled orthopedic device worn on a body of a patient fitted with the orthopedic device and influences movement by microprocessor-controlled actuation of actuators, the method comprising the following computer-implemented steps:
providing a digital communication link from a mobile terminal device to an adjustment database, in which at least one adjustment value for at least one parameter of the respective microprocessor-controlled actuation and associated patient data of a patient fitted with the respective orthopedic device are stored for a plurality of microprocessor-controlled orthopedic devices,
calculating at least one adjustment value of at least one parameter of the microprocessor-controlled activation of the orthopedic device of the patient depending on patient data from the patient whose microprocessor-controlled orthopedic device is to be adjusted, and/or depending on the adjustment values stored in the adjustment database and associated patient data, and
displaying the at least one calculated adjustment value on the mobile terminal device in order to adjust the microprocessor-controlled orthopedic device.

2. The method according to claim 1, wherein the patient data of the patient whose orthopedic device is to be adjusted is transmitted from the mobile terminal device to the adjustment database, wherein the at least one adjustment value of the at least one parameter is calculated by the adjustment database.

3. The method according to claim 1 wherein the at least one adjustment value is calculated as a function of the stored adjustment values that relate to the same orthopedic device or at least a same type or kind of device.

4. The method according to claim 1 wherein the at least one adjustment value is calculated as a function of the stored adjustment values that are no older than a given date.

5. The method according to claim 1 wherein the at least one adjustment value is calculated as a function of the stored adjustment values that relate to at least one orthopedic device that has performed a particular number of movements.

6. The method according to claim 1 wherein the at least one adjustment value is calculated as a function of the stored adjustment values where a change was made by an orthopedic professional, wherein the change is no older than a given date.

7. The method according to claim 1 wherein at least one correlation parameter is calculated by the adjustment database and transmitted to the mobile terminal device, wherein at least one adjustment value is calculated as a function of the patient data and the at least one correlation parameter, whether or not the even communication link has been provided.

8. The method according to claim 1 further comprising after adjusting the patient's orthopedic device, the current adjustment values are transmitted from the mobile terminal device to the adjustment database.

9. The method according to claim 1 wherein adjustment of the patient's orthopedic device with up-to-date adjustment values is performed by the mobile terminal device which establishes a communication link to the orthopedic device to provide the up-to-date adjustment values.

10. The method according to claim 1 further comprising providing a machine learning system which has learned a correlation of patient data of patients and information about their respective microprocessor-controlled orthopedic devices as input data with associated adjustment values of at least one parameter as output data, wherein the patient data of the patient and information on their orthopedic device is entered into the machine-learning system as input data and at least one calculated adjustment value is subsequently obtained as output data in order to calculate the at least one adjustment value for adjusting the patient's orthopedic device.

11. The method according to claim 10, wherein the machine learning system is an artificial neuronal network.

12. A computer program with program coding on a non-transient computer readable medium to carry out the method according to claim 1 when the computer program is run on a data processing system.

13. A system comprising an adjustment database and one or multiple mobile terminal devices (11), the system being configured to carry out the method according to claim 1.
